(19) United States
(12) Patent Application Publication
(10) Pub. No.: US 2011/0218436 A1
(43) Pub. Date: Sep. 8, 2011

(54) MOBILE ULTRASOUND SYSTEM WITH COMPUTER-AIDED DETECTION

(76) Inventors: Russell H. Dewey, Los Altos, CA (US); Kenneth Jacobsen, Fremont, CA (US)

(21) Appl. No.: 12/718,969

(22) Filed: Mar. 6, 2010

(51) Int. Cl. A61B 8/14

(52) U.S. Cl. 600/443

(57) ABSTRACT

A mobile ultrasound scanning system with computer-based detection methods is disclosed, comprising: (1) a small ultrasound probe containing at least one transmitting and receiving element; a probe enclosure (21); a human attachment means (22) for temporarily attaching the probe enclosure to a human finger, or group of fingers, or the palm of a human hand; (2) a system data communications means for data communications and control communications between the small ultrasound probe and a mobile data processing computer; (3) a mobile data processing computer containing a CPU, operating system, remote information network TCP/IP connectivity, and memory; (4) a library of down-loadable and installable computer-based medical-condition detection methods, which methods execute within the said mobile data processing computer, and which library contains at least one computer-based method capable of executing in said data processing computer and capable of analyzing ultrasound probe echo data and drawing probable inferences concerning the detection of a specific disease therefrom; (5) a user interface means for accepting end-user input commands and input data, and for displaying inference results derived from the execution of a computer-based method of disease detection, and presenting said results to the end-user as a simple color-coded visual indicator.

### BACKGROUND

1. Field of Invention

This invention relates to ultrasound-based systems for the diagnosis, testing and detection of medical conditions; specifically to a mobile ultrasound scanning system that includes a computer-based method for analyzing echo data acquired from an ultrasound probe and for drawing inferences from those data about the possible presence of a disease, and able to perform its functions without a traditional medical image display and without a trained medical professional operator.

2. Discussion of Prior Art

Ultrasound technology is generally recognized as a valuable tool for diagnosing numerous diseases and medical conditions. Until the present invention, however, it has simply been too expensive and difficult for use in remote or impoverished locations, or in developing countries, or by individuals anywhere who recognize the timeliness, convenience, and cost-effectiveness of frequent self examinations for early detection of health-related problems.

Two prior US patent citations were discovered that combine ultrasound technology with computer-aided detection.

In U.S. Pat. No. 4,470,304, Nusbickel, Jr, et al, Sep. 11, 1984, all Claims refer to “an ultrasonic inspection system for determining area defects in a flat workpiece movable along an inspection path . . . ” and includes specifications for visual imaging and computer printing of flaw maps. The current disclosed invention does not scan movable workpieces, does not produce visual images, does not produce computer printing of flaw maps, and requires no trained operator.

In U.S. Pat. No. 7,556,602, Wang, et al, Jul. 7, 2009, there is only one independent Claim and that Claim refers to a method for producing a plurality of visual images, or thin slices, of a compressed patient's breast and then assembling those thin slices into thick slices in order to present visual medical information of better quality to an assumed trained operator to examine and diagnose. The current disclosed invention does not produce visual images, requires no trained operator, and uses methods of medical-condition detection that do not require a breast to be “compressed”, and is not limited to breast cancer screening, as is implied by the title and abstract.

Searching further, in commercial products and current university projects, we learn that ultrasound systems in the prior art for health-care diagnosis, testing and detection typically include the following major system components: ultrasound probes, cables and wires, a data processing unit, imaging electronics, methods for interpretation and detection, and methods for communication of results.

Ultrasound Probes

Ultrasound systems in the prior art typically include an ultrasound probe designed to be grasped in the hand of a trained medical technician and carefully pointed at a specific spot on a patient's body in order to acquire ultrasound echo data. Such probes are well known in the art. For example, see: http://www.interson.com.

Prior art probes will be unsuitable, however, for self-examinations. In a self examination, the end-user needs to hold the probe by herself or himself, and needs to press the probe gently but comfortably against his or her body to perform a scan. The presently disclosed invention reduces the size and weight of prior art probes and re-designs the physical enclosures of prior art probes in order to make them temporarily attachable to an end-user's finger or multiple fingers or palm of the hand , and thereby make the probe more useful for self-examinations.

Cables and Wires

Ultrasound systems in the prior art typically include a cable for data communications and control signals, connecting a probe with a nearby data processing unit. This approach may be suitable in a medical clinic setting, where the cable can be pulled out of the way by the medical technician, but may prove to be unsuitable for consumers performing self examinations. In a self examination, the consumer would more likely prefer to not have a cable dragged across his or her body. The present invention discloses an alternative embodiment in which the awkward cable is replaced with a wireless connection between the probe and the data processing unit.

Data Processing Unit

Ultrasound systems in the prior art typically include a custom-designed and manufactured data processing unit that acquires a continuous feed of echo data from a probe via a fixed data communication cable, and then formats and organizes those data, and then produces a rapid series of displayable digital images of the data, typically measured in frames per second. The digital images are then viewed by a trained medical operator who looks at the images on the screen and interprets the results. All of this processing requires a data processing device with considerable processing power. Recently, some manufacturers have begun substituting commercial off-the-shelf desktop computers, notebook computers and mobile phones and PDAs for the previously custom-made parts. For example, see any of these contemporary research and commercial products:

http://research.microsoft.com/en-us/collaboration/focus/health/msr_ultrasound.pdf
http://www.hojohnlee.com/weblog/archives/2005/11/15/low-cost-portable-ultrasound-probe-for-notebook-computers/
http://www.innovasound.us/Medical%20Site/medical_index.html

The presently disclosed invention is also focused on small, mobile data processing devices, including Smartphones, but with a key difference. The presently disclosed invention eliminates the need for a high-resolution multi-frame display of ultrasound echoes and thereby eliminates the need for data processing capabilities and display electronics needed to produce such a display and present it to an end-user.

Imaging Electronics

Prior art for ultrasonic systems for diagnosis, testing and detection typically include electronic circuits and methods for creating visual displays, some in three-dimensions, of the echo data received by the ultrasound probe. Many of the current commercial vendors, including those cited herein, have moved imaging electronics into the probe itself, thereby making the probe larger than it needs to be and heavier. It should be obvious that displaying an ultrasound image on a Smartphone screen, at low resolution and slow frame rates, is unlikely to provide sufficient visual detail for an accurate human-generated diagnosis or detection of disease. In the presently disclosed invention, the imaging electronics and methods are eliminated, and replaced with specific methods of computer-aided detection, thereby allowing for a re-design of the probe to make it more suitable for self-examinations.

Computer-Aided Methods for Interpretation and Detection

Prior art assumes that ultrasound scans will be operated by and the results will be interpreted by medically trained personnel who will then verbally communicate the results to the patient whose body was scanned. It should be readily apparent that this scenario is unsuitable for self-examinations. The presently disclosed invention replaces the trained medical technician as the provider of interpretation and detection with computer-aided methods of disease detection, which methods embody and encapsulate a substantial portion of the expertise of a trained medical technician.

In U.S. Pat. No. 5,212,637, Saxena, May 18, 1993, a method is disclosed for investigating mammograms for masses and calcifications. The method, in claim 1, is comprised of four steps: (a) converting intuitive criteria (from trained radiologists) into numerical (statistical) criteria; (b) programming a computer with said (statistical) criteria; (c) acquiring information in said computer program defining a human breast; (d) using said computer to . . . identify regions to be investigated (by an assumed radiologist) . . . said information defining a calcification (number, size and shape). Dependent Claims all refer to acquiring said data (step c) by optoelectronic means (digital photographs of a film). In the present disclosed invention, a computer-aided detection method is disclosed that uses steps (a) and (b); but differs in steps (c) and (d). The presently disclosed invention acquires information by directly downloading ultrasound probe data from a probe into a data processing unit, and does not identify spatial regions to be investigated by a radiologist. Alternative embodiments of the present invention may replace the statistical model in steps (a) and (b) with a rule-based expert system, or by a Bayesian Belief network, or other more modern methods of computer-aided intelligent reasoning. In alternative embodiments of the present invention, the subject being examined is not necessarily a human breast and the condition being tested is not necessarily calcification and masses. The apparatus disclosed in the '637 Patent requires a digital camera to capture data from a mammogram film, a table to hold said film, and a means for outputting information defining a region of the target breast. The currently disclosed patent requires no such apparatus, as the data is downloaded directly from an Ultrasound probe into a mobile data processing unit and the result is displayed as a simple color-coded indicator on the mobile data processing device.

Data Quality and Calibration

One of the challenges for effective computer-aided detection methods is the quality of the data input to the detection method. It does not matter how good the computer-aided method is, if the input data is not robust. Some of the external factors affecting data quality include: skin color, skin thickness, tissue and muscle size, and tissue and muscle density. End-users will certainly have differences in those factors. Those differences suggest a need for calibration of the system so that the probe will be able to detect usable ultrasound echos and convert those echos into quality-assured digital data for the computer-aided method to use. To address this problem of data quality and calibration, the present invention discloses a user interface that includes a calibration means for adjusting the behavior of the system to accommodate physical differences in the end-user that would otherwise affect ultrasound performance.

Methods for Communication of Results

In the prior art, the results of an ultrasound scan are typically communicated verbally to the patient by the trained medical technician. That patient may or may not fully understand what the medical technician did or said. In contrast, the presently disclosed invention communicates the results of a scan directly to the end-user by means of a simple color code that can be readily understood by anyone, regardless of their education and regardless of their spoken language.

In summary, the present invention improves on the prior art of ultrasound systems in many ways and discloses a system that is oriented around small size and mobility, can be built at lower cost, and includes computer-based detection methods for medical condition-specific self-examinations—in contrast with prior art computer-based methods intended to assist a medical technician to visually evaluate a digital image. The present invention, in its preferred embodiment, eliminates the professional medical personnel; eliminates the need for a digital image monitor; eliminates the typically expensive dedicated data processing device; moves data acquisition, formatting and organization to a mobile hand-held computing device; includes a library of down-loadable and installable computer-aided detection software that is medical condition specific; and communicates the results of the self-exam as a simple color-coded display—one color meaning “Nothing abnormal found” and a second color meaning “Something suspicious found; See your doctor.”.

### OBJECTS AND ADVANTAGES

The present invention has a number of advantages over the prior art, including the following:

1) Personal and private: The computer-assisted self-examination and detection steps can be performed at home, by the patient alone, outside the presence of additional people.

2) Convenient and time-saving: The patient does not need to travel to a medical facility to have the examination and detection steps performed. The scan and detection results can be performed at any place and at any time of night or day.

3) Lower Cost: The patient eliminates the cost of transportation, insurance deductibles, co-payments, and other charges associated with having an ultrasound scan or film scan performed at a medical facility. The service providers and insurance companies save money by having only medically necessary visits to the medical facilities; self-examination scans are done at the patient's home.

4) Easy to use: No training is required to use the system, and a two-color output display is understandable in all of the world's languages.

5) Mobile: Because so many people already have Smartphones, and other mobile data processing devices such as PDAs, pocket computers, and multimedia devices, and because the hand-held probe is itself small, the entire system can be readily taken by a consumer from place to place.

6) Specific in Purpose: By selecting and then loading a specific computer-based detection method into the hand-held mobile device, from a library of available detection methods, the user may do a self-exam specifically tailored to the early detection of a specific disease—such as breast masses, testicular cancer, COPD and emphysema, thyroid enlargements, etc. In this way, each self-examination can be tailored to look for early evidence of specific malignancies.

7) Individual: Adjusting its computer-aided detection methods to accommodate end-users with different physical characteristics.

### DRAWINGS FIGURES

FIG. 1a is a simplified functional block diagram illustrating the primary functional components of the preferred embodiment of the present invention.

FIG. 1b is a simplified block diagram of an alternative embodiment of the present invention in which an ultrasound probe has been equipped with wireless communications capabilities.

FIG. 1c is a simplified block diagram of an alternative embodiment in which most components are contained in a single device enclosure.

FIG. 1d is a simplified block diagram of an alternative embodiment in which both a library of detection methods and a data processing computer are located remotely in an information processing network, such as the World Wide Web, while a probe and user interface are contained in a single hand-held enclosure.

FIG. 2 is a simplified flow chart illustrating the operation of the preferred embodiment of the present invention.

### SUMMARY

A mobile ultrasound scanning system with computer-based detection methods is disclosed, comprising: (1) a small ultrasound probe containing at least one transmitting and receiving element; a probe enclosure (21); a human attachment means (22) for temporarily attaching the probe enclosure to a human finger, or group of fingers, or the palm of a human hand; (2) a system data communications means for data communications and control communications between the small ultrasound probe and a mobile data processing computer; (3) a mobile data processing computer containing a CPU, operating system, remote information network TCP/IP connectivity, and memory; (4) a library of down-loadable and installable computer-based medical-condition detection methods, which methods execute within the said mobile data processing computer, and which library contains at least one computer-based method capable of executing in said data processing computer and capable of analyzing ultrasound probe echo data and drawing probable inferences concerning the detection of a specific disease therefrom; (5) a user interface means for accepting end-user input commands and input data, and for displaying inference results derived from the execution of a computer-based method of disease detection, and presenting said results to the end-user as a simple color-coded visual indicator.

### DESCRIPTION OF INVENTION—PREFERRED EMBODIMENT

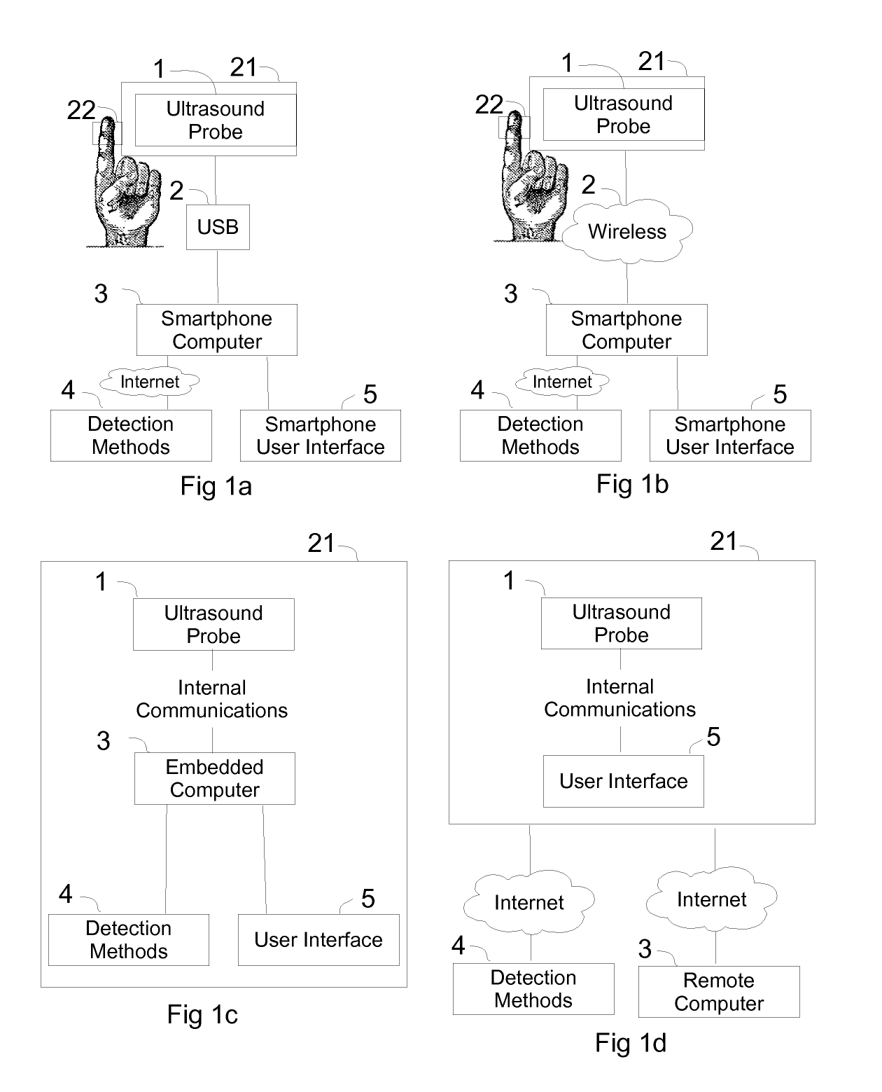

FIG. 1a is a simplified functional block diagram illustrating the primary functional components of the preferred embodiment of the present invention. For the sake of simplicity, wiring inter-connections, power supplies, cable connectors, device drivers, and power management components are not shown, and will be readily apparent to anyone skilled in the art.

The rectangle labeled 1 represents a small ultrasound probe containing at least one transmitting and receiving element and which is contained within an enclosure 21 that includes a probe attachment means 22 for temporarily attaching the probe enclosure to a human finger, or to a group of fingers, or to the palm of a human hand.

The rectangle labeled 2 represents a system data communications means for data communications and control communications between the probe and a data processing computer. In the preferred embodiment of the present invention the system data communication means is a Universal Serial Bus (USB) cable. Alternative embodiments are also presented in FIGS. 1b, 1c, and 1d.

The rectangle labeled 3 represents a mobile data processing computer. In the preferred embodiment of the present invention the mobile data processing computer is a system comprised of a central processing unit, an operating system, a means for remote information network TCP/IP connectivity, and memory and is located within a Smartphone of a type that is readily known within the art. A Smartphone is an electronic handheld device with a self-contained power source that integrates the functionality of a mobile phone, personal digital assistant (PDA) or other information appliance. Many Smartphones include the functionality of an Internet web browser.

See: http://en.wikipedia.org/wiki/Smartphone

The rectangle labeled 4 represents a library containing at least one computer-based method capable of being selected and downloaded from its location, installed into said data processing computer and, once loaded, capable of being executed, whereby it analyzes ultrasound probe data and draws probable inferences concerning disease detection therefrom. In the preferred embodiment of the present invention the library of down-loadable and installable computer-based methods of disease detection is located at an information network location, remote from the data processing computer 3. In the preferred embodiment of the present invention, the library of computer-based methods of disease detection contains at least one method tailored to interpreting ultrasound data and detecting the presence of a specific disease. Since many computer-aided detection methods exist in the art, and in the interest of brevity, the details of such a method are not included here. In the preferred embodiment of the present invention, the method used is an adaptation of the method disclosed in U.S. Pat. No. 5,212,637 (Saxena 1993).

The rectangle labeled 5 represents a user interface means for accepting user commands and for displaying inference results derived from the execution of a computer-based method of disease detection. The results will be displayed as a simple color-coded visual indicator. The user input/output means may also display the status of the operation of the disclosed ultrasound scanning system. In the preferred embodiment of the present invention the user interface means is a touch-sensitive display screen of a type typically found on mobile data processing devices such as Smartphones. Unlike prior art, the present invention does not create a human-readable image of scan data. Only the likelihood of an abnormal condition being present is shown.

The rectangle labeled 21 is an enclosure of unspecified type containing the ultrasound probe 1 and probe transmitting and receiving electronics. The rectangle labeled 22 is a human attachment means for temporarily attaching the probe enclosure to a human finger, or group of fingers, or the palm of a human hand. In the preferred embodiment of the present invention the human attachment means is an adjustable-size cincture fabricated from elastic materials. In an alternative embodiment the human attachment means cincture is fabricated from materials with mechanical size-adjusting properties.

### OPERATION OF INVENTION—PREFERRED EMBODIMENT

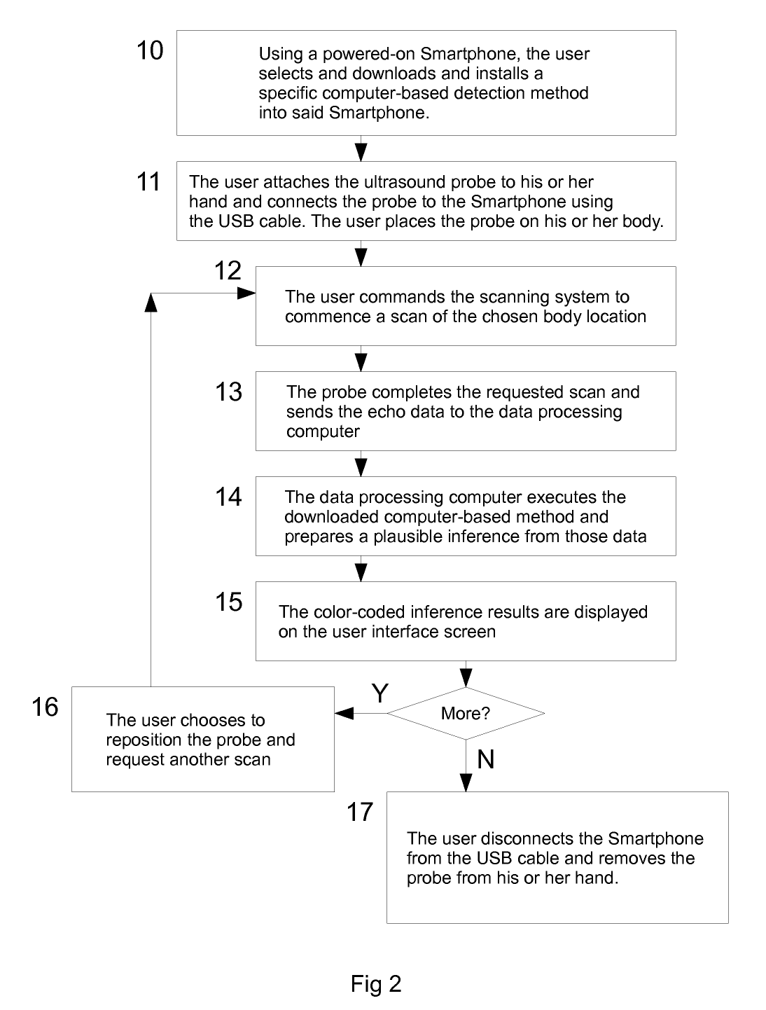

FIG. 2 is a simplified flow chart summarizing the operation of the preferred embodiment of the present invention, comprising the following primary steps:
10 Using a powered-on Smartphone, the user selects and downloads and installs a specific computer-based detection method into said Smartphone.
11 The user attaches the ultrasound probe to his or her hand and connects the probe to the Smartphone using the USB cable. The user selects calibration options and places the probe on a part of his or her body.
12 Using the Smartphone user interface, the user commands the scanning system to commence a scan of the chosen body location
13 The probe completes the requested scan and sends the echo data to the data processing computer located within the Smartphone
14 The data processing computer executes the downloaded computer-based method and prepares a plausible inference from those data
15 The color-coded inference results are displayed on the user interface screen
16 The user chooses to either reposition the probe and request another scan, or
17 The user disconnects the Smartphone from the USB cable and removes the probe from his or her hand.

### DESCRIPTION OF INVENTION—ALTERNATIVE EMBODIMENTS

In alternative embodiments of the present invention:

The system data communications means for data communications and control communications between the small ultrasound probe (1) and a mobile data processing computer (2) may use wireless technology rather than the USB cable as described. Wireless data and control communication technologies, such as BlueTooth and WiFi, are well known in the art.

The mobile data processing computer (3) may be a PDA, or desktop personal computer, or mobile notebook computer, or multi-media device with Internet and computer processing capabilities. The data processing computer (3) may also be a service hosted on a computer system server located at an Internet-based private or public hosting service.

The library containing at least one computer-based method capable of executing in said data processing computer and capable of analyzing ultrasound probe data and drawing probable inferences concerning disease detection therefrom (4) may be located at an Internet-based commercial service or at a private Internet-based server, or may be located on removable media such as CD-ROM or DVD or flash memory devices.

The user interface means (5) for accepting user commands could be a keyboard or custom button switches on the probe itself. The user interface means for displaying inference results derived from the execution of a computer-based method of disease detection as a simple color-coded visual indicator (5) could instead contain a variable sized graphic to represent the quantitative level of belief in the likelihood that a particular disease is present.

The human attachment means for attaching the probe to a finger, or group of fingers, is of a type of attachments classified as cinctures, and may be made of any material that permits the adjustment of the size of the cincture through either elastic or mechanical methods. The human attachment means for attaching the probe to the palm of a hand is of a type comprised of a combination of a glove that slips on and off the hand and a glove attachment means for permanently or temporarily attaching the probe to said glove.

### CONCLUSION, RAMIFICATIONS, AND SCOPE

Accordingly, the reader will see that the mobile ultrasound scanning system with computer-based detection methods can be used to make health care self examinations more effective for consumers, as well as for health care practitioners in poor countries or communities that lack financial resources for more expensive systems. The ultrasound apparatus and method for health care self-examinations disclosed in the present invention has other advantages as well: unlike current art, it is mobile; it saves time and money for the consumer and for the health care providers and insurers; it is application specific—that is, there is one personal ultrasonic computer-based method for detection of breast masses and calcification, and another for testicular masses and calcification, another for thyroid conditions, and so forth. Except for the computer-based methods, the system can be manufactured primarily out of off the shelf commercial components, together with a few custom electronic interfaces and device handlers.

In contrast with prior art, the disclosed specific combination of ultrasound components, electronic components, specialized enclosures, mobile data processing devices, communication technologies, computer-based disease detection methods, and simplified visual results is both novel and non-obvious.
1. The disclosed invention has results that are superior to prior art in terms of cost, size, convenience, privacy, understandability, and specificity. No one has done exactly this before.
2. The disclosed invention solves a problem that was never before even recognized. This will be the first commercial product that delivers an intelligent product for the masses of people around the world, which is capable of low-cost ultrasound self-examination and early-stage detection of specific diseases—without the need for medially trained personnel.
3. The disclosed invention goes a long way in solving a long-felt, long-existing, but unsolved need to lower the costs of health-care delivery and to increase the effectiveness of early detection.
4. The disclosed invention will create an entirely new industry; an industry designing and manufacturing ultrasound self-examination devices, and an industry designing and producing intelligent methods for detecting specific diseases from evidence contained within unfiltered and undisplayed ultrasound echo data.

Although the description above contains many specifications, these should not be construed as limiting the scope of the invention but as merely providing illustrations of some of the presently envisioned embodiments of this invention. For example, the ultrasound probe could have a single element, or an array of elements; the communication means could be a cable, such as a USB cable, or the communication means could be wireless, such as Bluetooth or WiFi; the mobile data processing device could be a Smartphone, or a PDA, or a pocket computer, or a multimedia device with computer and communications capabilities, or a common notebook or tablet personal computer; the CAD computer program method for detecting mammary masses and calcification could be statistical based, or it could be based on expert rules, or a neural network, or a Bayesian network of conditional probabilities; other computer-based methods could be written for other medical self-examinations. Thus the scope of the invention should be determined by the appended claims and their legal equivalents rather than by the examples given.

What is claimed is:

1. A mobile ultrasound scanning system with computer-based detection methods, that is comprised of:
(a) a small ultrasound probe comprised of at least one ultrasound transmitting element and at least one ultrasound receiving element, electronic circuits needed for ultrasound transmitting and receiving, and a probe enclosure containing said transmitting elements, receiving elements, and electronic circuits;
(b) a human attachment means for temporarily attaching said probe enclosure, and its contents, to a human hand;
(c) a system data communications means for data communications and control communications between the small ultrasound probe and a mobile data processing computer;
(d) a mobile data processing computer containing a CPU, operating system, remote information network connectivity, and memory;
(e) a library of down-loadable and installable computer-based methods containing at least one computer-based method capable of executing in said data processing computer and capable of analyzing ultrasound probe echo data and drawing probable inferences concerning disease detection therefrom; and
(f) a user interface means for accepting end-user input commands and input data, and for displaying inference results derived from the execution of a computer-based method of disease detection as a simple color-coded visual indicator.

2. The system of claim 1 wherein said data processing computer is a data processing device contained within a Smartphone, which is a mobile telephone containing a programmable computer that runs operating system software, processes data, connects to Internet sites, runs application software, and provides a standardized interface and platform for developers.

3. The system of claim 2 wherein said system data communication means is a Universal Serial Bus (USB) cable and connectors.

4. The system of claim 3 wherein said inference results are displayed as a variable sized graphic to represent the quantitative level of belief in the likelihood that a particular disease is present.

5. The system of claim 2 wherein said system data communication means is a wireless technology, of a class of wireless technologies including BlueTooth and WiFi.

6. The system of claim 5 wherein said inference results are displayed as a variable sized graphic to represent the quantitative level of belief in the likelihood that a particular disease is present.

7. The system of claim 1 wherein said data processing computer is any mobile data processing device of a class of computer devices including notebook computers, pocket-sized computers, multi-media devices, and Personal Digital Assistants (PDAs).

8. The system of claim 7 wherein said data communication means is a standard Universal Serial Bus (USB) cable and connectors.

9. The system of claim 8 wherein said inference results are displayed as a variable sized graphic to represent the quantitative level of belief in the likelihood that a particular disease is present.

10. The system of claim 5 wherein said data communication means is a wireless technology, of a class including BlueTooth and WiFi.

11. The system of claim 10 wherein said inference results are displayed as a variable sized graphic to represent the quantitative level of belief in the likelihood that a particular disease is present.

12. The system of claim 1 wherein said small ultrasound probe, and system data communication means, and data processing computer, and user interface means are contained within a single system enclosure of a size about equal to the size of a semi-open human hand, which system enclosure is capable of wireless communicating with said library of computer-based detection methods for selecting and downloading and installing specific methods from said library.

13. The system of claim 12 wherein the results of a scan are displayed as a variable sized graphic to represent the quantitative level of belief in the likelihood that a disease is present

14. The system of claim 1 wherein said small ultrasound probe, and system data communications means, and user interface means are all contained within a single system enclosure capable of wireless communicating with a data processing computer in a remote information network, and capable of wireless communicating with a library of computer-based detection methods in a remote information network.

15. The system of claim 14 wherein the results of a scan are displayed as a variable sized graphic to represent the quantitative level of belief in the likelihood that a disease is present.

16. The system of claim 1 wherein the human attachment means is an adjustable-size cincture capable on encircling one or more fingers on a human hand.

17. The system of claim 1 wherein the human attachment means is comprised of a slip-on and slip-off flexible finger enclosure in the shape of a sewing thimble, and a finger enclosure attachment means for attaching the probe enclosure to said finger enclosure.

18. The system of claim 1 wherein the human attachment means is comprised of a slip-on and slip-off flexible glove enclosure in the shape of a glove for a human hand, and a glove attachment means for attaching the probe enclosure to said glove enclosure.

19. The system of claim 1 wherein the user interface means includes a plurality of touch-sensitive colored rectangles, where each of the said rectangles corresponds to a plurality of programmable numerical values, where said numerical values may be read by and used by an installed computer-aided detection method to modify its execution logic based on said numerical values, and thus taking into account different physical characteristics of different end-users.
